(19) United States
(12) Patent Application Publication
(10) Pub. No.: US 2022/0241006 A1
(43) Pub. Date: Aug. 4, 2022

(54) TREATMENT DEVICE

(71) Applicant: OLYMPUS MEDICAL SYSTEMS CORP., Tokyo (JP)

(72) Inventors: Akira MIYAZAKI, Yokohama-shi (JP); Ryu ONUMA, Tokyo (JP); Ojiro KITAMURA, Tokyo (JP)

(73) Assignee: OLYMPUS MEDICAL SYSTEMS CORP., Tokyo (JP)

(21) Appl. No.: 17/542,641

(22) Filed: Dec. 6, 2021

Related U.S. Application Data
(60) Provisional application No. 63/145,237, filed on Feb. 3, 2021.

(51) Int. Cl. A61B 18/14

(52) U.S. Cl. CPC A61B 18/1445; A61B 2018/0063

(57) ABSTRACT

A treatment device equipped with high frequency capability has a pair of clasping jaws. The upper and lower jaws are openable-closable and serves as high frequency electrodes to clamp and seal and/or transect tissue. A blade traverses a channel in the upper and lower jaws and is used to open and close the upper and lower jaws. Additional structures and mechanisms contribute to the operation of the upper and lower jaws and add strength to the opening of the clasping jaws, which is useful in the procedure of peeling tissues using the opening movement of the clasping jaws.

### RELATED APPLICATION DATA

This application is based on and claims priority under 35 U.S.C. § 119 to U.S. Provisional Application No. 63/145,237, filed Feb. 3, 2021, the entire contents of which are incorporated herein by reference.

### FIELD OF DISCLOSURE

The present disclosure relates to a treatment device having an openable-closable first and second jaws. The openable-closable jaws serve to clamp tissues for sealing and transecting. A blade is used to close the jaws and various mechanisms are disclosed for assisting the opening of the jaws, which is useful in the procedure of peeling tissues.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

FIGS. 14A, 14B, and 14C are figures of a treatment device disclosed in the related art (United States Patent Application Publication No. 2009/0076506A1). FIG. 14A shows a working end 210 with jaws 222a and 222b in an opened position with outward-facing surfaces 262A and 262B and energy-delivery surfaces 265A and 265B. A channel 242 within the jaws accommodates the movement of reciprocating member 240, which may comprise a tissue-cutting element, for example by having a sharp distal edge. The edges 268 of energy-delivery surfaces 265A and 265B may be rounded to prevent the dissection of the tissues. FIG. 14B shows a working end 210 with jaws 222a and 222b in a closed position. The channel 242 where reciprocating member 240 is designed to move back and forth is visible, as well as dimension D between the energy-delivery surfaces. FIG. 14C shows the distal end of reciprocating member 240 having upper and lower flanges or “c”-shaped portions 250A and 250B. The flanges 250A and 250B respectively define inner cam surfaces 252A and 252B for slidably engaging outward-facing surfaces 262A and 262B of jaws 222a and 222b.

A drawback of the related art treatment device is that, although reciprocating member 240 serves to close the jaws 222a and 222b by sliding the reciprocating member 240 progressively forward through the channel 242, moving back the reciprocating member 240 provides a weak force to open the jaws 222a and 222b, which may not enough force to open the jaws 222a and 222b when there is a resistance. When the operators are required to open the jaws of the treatment device during the peeling procedure despite the resistance of any resisting objects, the related art treatment device would not be able to accomplish that task due to the lack of force for opening the jaws 222a and 222b.

### SUMMARY

Accordingly, there is a need for designing a treatment device with an improved structure to increase the forces that can be applied to open the jaws of the treatment end, which would substantially obviate one or more of the issues due to limitations and disadvantages of related art treatment device.

An object of the present disclosure is to provide an improved treatment device that provides an efficient design for the opening and closing of the upper and lower jaws serving as high frequency electrodes compared to the related art. At least one or some of the objectives is achieved by the treatment device disclosed herein.

Embodiments of the disclosed treatment device comprises a treatment device with a body including a movable handle and a connection configured to connect to a power source supplying power for conducting a high-frequency treatment with the treatment device, a longitudinally extending shaft having a proximal end connected to the body, and a treatment end pivotally joined to a distal end of the shaft. The treatment end includes a movable cutting blade, a jaw structure having a first jaw and a second jaw, and a first channel in the first jaw and a second channel in the second jaw. The first jaw and the second jaw are connected at a base end by a joint structure to pivot the first jaw relative to the second jaw to open and close the jaw structure. When the jaw structure is in a closed state, the first channel and the second channel form a route for a cutting blade to move back and forth longitudinally between the base end of the jaw structure and a distal end of the jaw structure in response to a first input at the body. A force is applied through means to move the first jaw away from the second jaw in response to a second input at the body.

Embodiments of the disclosed treatment device further comprises a treatment device with wire connected to one of the first jaw or the second jaw to apply the force to move the first jaw away from the second jaw in response to the second input at the body.

Embodiments of the disclosed treatment device further comprises a treatment device with a hooking member protruding from the movable cutting blade in combination with a member protruding from one of the first jaw or the second jaw serving to apply the force to move the first jaw away from the second jaw in response to the second input at the body. During a retraction of the movable cutting blade, the hooking member contacts the member protruding from one of the first jaw or the second jaw and applies the force to move the first jaw away from the second jaw.

Embodiments of the disclosed treatment device further comprises a treatment device with rear surface of the movable cutting blade in combination with a member protruding from one of the first jaw or the second jaw serving to apply the force to move the first jaw away from the second jaw in response to the second input at the body. During a retraction of the movable cutting blade, the rear surface of the cutting blade contacts the member protruding from one of the first jaw or the second jaw and applies the force to move the first jaw away from the second jaw.

Embodiments of the disclosed treatment device further comprises a treatment device with a member connected to the inner ceiling of one of the first jaw or the second jaw with an elastic member, such as a spring, in combination with a sloped surface formed on the movable cutting blade serving to apply the force to move the first jaw away from the second jaw in response to the second input at the body. During a retraction of the movable cutting blade, the sloped surface engages with the protruding from one of the first jaw or the second jaw to apply the force to move the first jaw away from the second jaw.

Embodiments of the disclosed treatment device further comprises a treatment device wherein the elastic member described above is a spring.

Embodiments of the disclosed treatment device further comprises a treatment device wherein the second input described above is a movement of the movable handle.

Embodiments of the disclosed treatment device further comprises a treatment device wherein the movable cutting blade described above includes a slope on its surface.

Embodiments of the disclosed treatment device further comprises a treatment device wherein a slider is placed between the movable handle and the first and second jaws.

Embodiments of the disclosed treatment device further comprises a treatment device wherein at least one of first and second jaws serves as a high frequency electrode.

Embodiments of the disclosed treatment device further comprises a treatment device wherein the movable cutting blade is a different pole electrode compared to at least one of the first and second jaws.

Embodiments of the disclosed treatment device further comprises a treatment device wherein a joint structure is placed between the shaft and the first and second jaws.

Embodiments of the disclosed treatment device further comprises a treatment device wherein the joint structure includes at least a pulley or a link.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 illustrates a treatment device including a body, a shaft, and a treatment end.

FIG. 2 illustrates an enlarged view of the treatment end shown in FIG. 1 and showing the upper and lower jaws in an open configuration.

FIGS. 3A and 3B illustrate the joint structure of the jaws and the joint structure between the treatment end and the shaft.

FIG. 4 illustrates in cross sectional view the upper and lower jaws of the treatment end and its internal structures.

FIG. 5 illustrates features of the upper jaw and the upper channel used to move the I-shaped cutting blade.

FIG. 6 illustrates the cross-sectional schema of the upper and lower jaws of the treatment end in its closed state.

FIGS. 7A and 7B illustrates in cross sectional view the upper and lower jaws of the treatment end and its internal structures.

FIG. 8A illustrates features of the inner view of the upper jaw and the upper channel used to move the I-shaped cutting blade.

FIG. 8B illustrates, in a back-perspective view, features of the upper and lower jaws and the structures used for opening the upper jaw.

FIG. 9A illustrates in cross sectional view the upper and lower jaws of the treatment end in an opened state.

FIG. 9B illustrates features of the upper and lower jaws and the structures used for opening the upper jaw.

FIG. 10 illustrates features of the upper and lower jaws and the structures used for opening the upper jaw.

FIGS. 11A, 11B, and 11C illustrates the upper and lower jaws in open and closed positions (FIG. 11A and FIGS. 11B and 11C, respectively) and structures used for closing the upper jaw.

FIG. 12 illustrates the cross-sectional schema of the upper and lower jaws of the treatment end in its closed state.

FIGS. 13A, 13B, and 13C illustrates the upper and lower jaws in closed and open positions (FIG. 13A and FIGS. 13B and 13C, respectively) and structures used for opening the upper jaw.

FIGS. 14A to 14C are figures of a treatment device disclosed in the related art.

For ease of viewing, in some instances only some of the named features in the figures are labeled with reference numerals.

### DETAILED DESCRIPTION

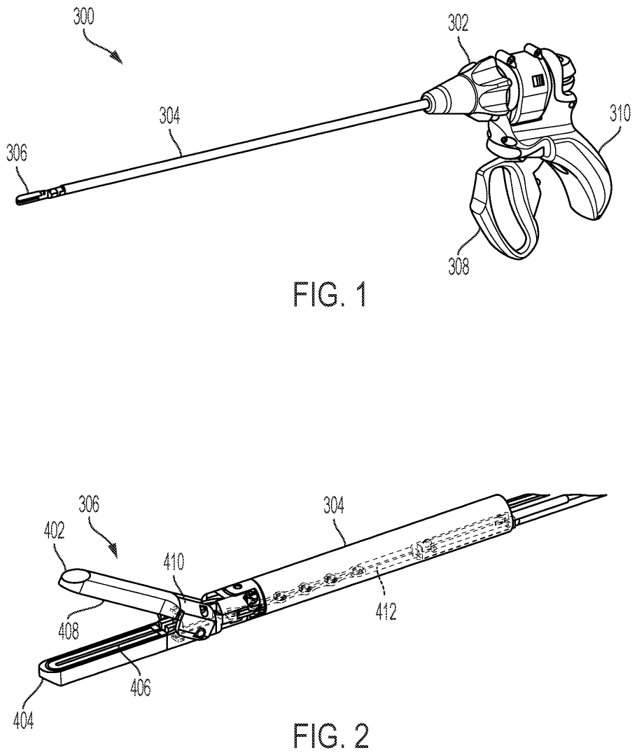

FIG. 1 is an illustration of a treatment device 300 including a body 302, a shaft 304, and a treatment end 306. The body 302 includes a moving arm 308 and a grip 310. The moving arm 308 is used to actuate and operate the functions of treatment end 306. The grip 310 is connected to a power source supplying power used for high-frequency treatment of treatment device 300. The power source can be a wired or wireless power source. The shaft 304 protects the wires and members within, necessary for operating the functions of treatment end 306.

FIG. 2 is an illustration of the treatment end 306 including the upper jaw 402 and lower jaw 404. The upper jaw 402 and lower jaw 404 may serve as electrodes electrically conducting the power supplied from the body 302 through the shaft 304. When the upper jaw 402 and lower jaw 404 are in a closed position, high frequency currents would be conducted between the jaws, and any object clasped between the jaws would either be dissected or sealed using the conducted high-frequency currents.

Both the upper jaw 402 and lower jaw 404 includes channels for moving the cutting blade 410, which may also serve as a high frequency electrode. The upper channel 406 and lower channel 408 is used as a route for the cutting blade 410 to move back and forth longitudinally between a base end of the jaws, i.e., the end connected for pivoting the jaws open and closed, and a distal end of the jaws. Moving the cutting blade 410 back and forth in the channels also serves to open and close the jaws, such as be displacing the upper jaw 402 relative to the bottom jaw 404, which may be stationary attached to the body of the treatment end 306. The operation members 412 goes through the shaft 304 and used for operating the functions of the treatment end 306. The operation members 412 can take any suitable form, such as cables and pulleys or other connection system that transmits input to control the operation of features of the treatment device 300, particular features associated with the treatment end 306.

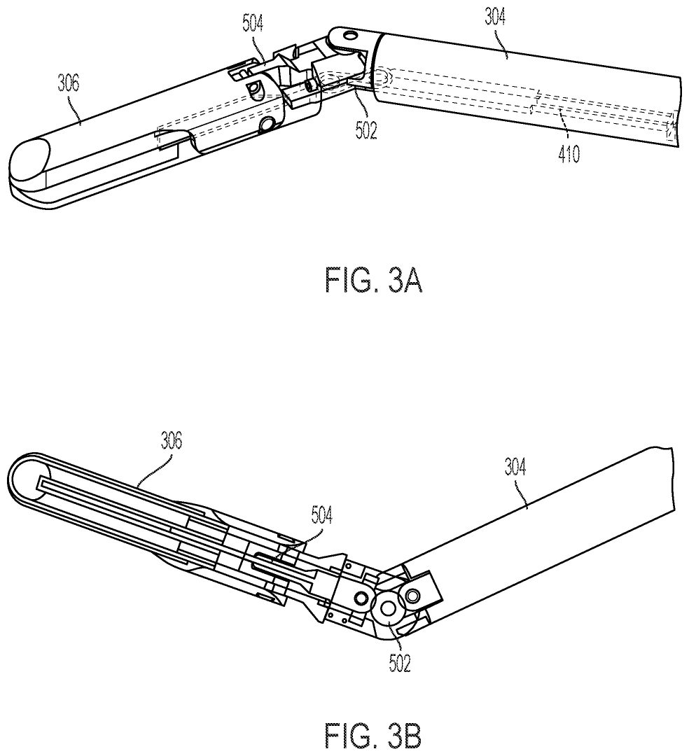

FIGS. 3A and 3B illustrate a joint structure 502 that is formed between shaft 304 and treatment end 306. The joint structure 502 serves to enable angular manipulation of the treatment end 306, enhancing the usability of the treatment device 300. The downside of having the joint structure 502 is that the added complexity in the structure serves as obstacles for the operation members 410 to pass through and effectively convey the operation movements to the treatment device 300. FIGS. 3A and 3B also illustrate features of the joint structure 504 of the jaw, which allow opening and closing of the jaws during operation.

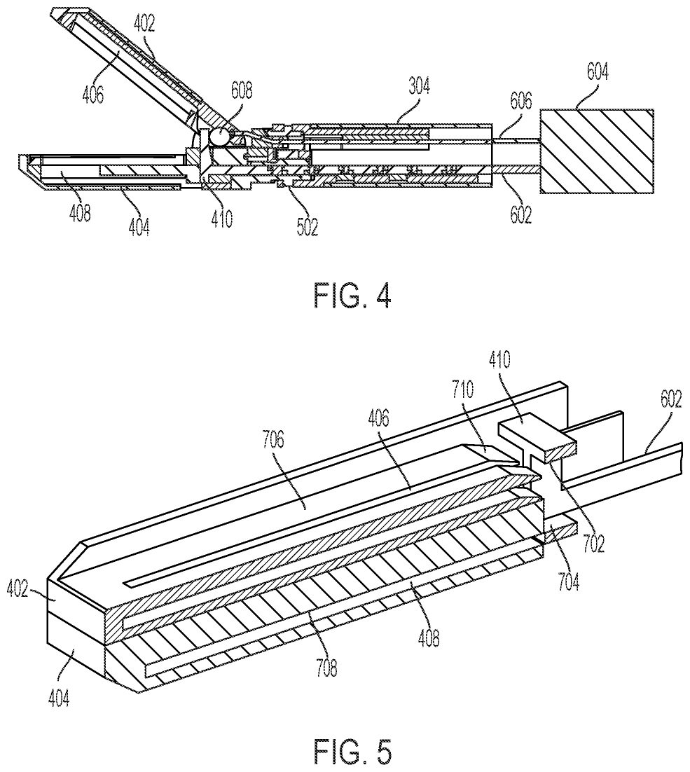

FIG. 4 illustrates in cross sectional view the upper and lower jaws of the treatment end 306 and its internal structures, including the mechanism for opening and closing the upper jaw 402 and lower jaw 404. The cutting blade 410 is connected to the cutting blade member 602 that goes through shaft 304 and connecting to the slider 604. The slider 604 is operable through movable arm 308 or other operating structures such as motor mechanisms. When the upper jaw 402 and lower jaw 404 are closed, the upper channel 406 and the lower channel 408 form a space in which the cutting blade 410 moves as the cutting blade member 602 is progressively pushed by operation of the slider 604. The cutting blade 410 travels within the space and can traverse back and forth between a first position adjacent the base end of the jaws and a second position at a distal end of the jaws. When there is an object located in the noted space between the upper jaw 402 and lower jaw 404, the cutting blade 410 traversing the upper channel 406 and the lower channel 408 serves to dissect such object using its blade formed on its distal end. The cutting blade 410 also has structures that cooperate with features of the upper channel 406 and the lower channel 408 to operate to close the upper jaw 402 using the structural design of the upper channel 406, which is further described herein. The upper jaw is connected to an upper jaw pulling wire 606, which is also connected to the slider 604. In response to the operation of the slider 604 to pull the upper jaw pulling wire 606, the upper jaw 402 is pulled away from the lower jaw 404 using the rotating fulcrum 608 placed at the base end of the upper jaw 402. The pulling of the upper jaw pulling wire 606 would occur simultaneously with pulling of the cutting blade member 604, which serves to pull back the cutting blade 410.

FIG. 5 is an inner view illustration of the upper jaw 402 and lower jaw 404. The I-shaped cutting blade 410 has a downward pushing surface 702 and an upward pushing surface 704 connected to the cutting blade member 602. The downward pushing surface 702 and the upward pushing surface 704 come in contact with the corresponding upper surface 706 and lower surface 708 of the upper and lower channels respectively when the cutting blade 410 goes through said channels. The downward pushing surface 702 initially contacts sloped surface 710 placed at the proximal end of the upper surface 706 and gradually slides past the sloped surface 710, thereby gradually pushing down the upper jaw 402 towards the lower jaw 404 to cause the upper jaw 402 to move toward the lower jaw 404. Although upward, downward, upper and lower are used herein, it is readily understood that these terms are in relation to the orientation depicted in the drawings and that a change in orientation does not change the position, operation and interoperability of features so described.

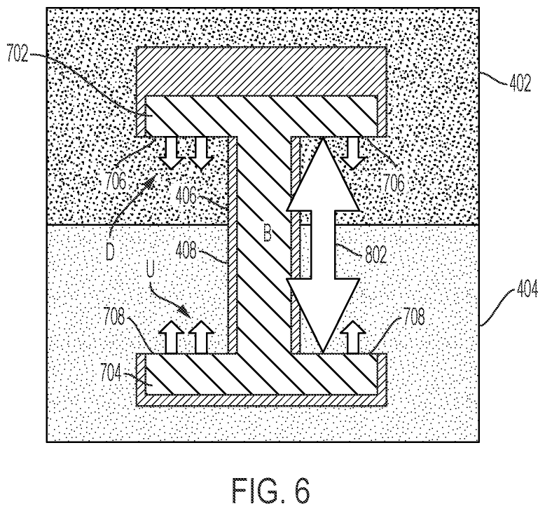

FIG. 6 is a cross sectional schema of the upper jaw 402 and lower jaw 404 where the jaws are in the closed state. The height 802 of the blade portion B of the I-shaped cutting blade 410 is equivalent (within manufacturing tolerances) to the distance between the upper surface 706 of the upper channel 406 and the lower surface 708 of the lower channel 408 when the upper jaw 402 and lower jaw 404 are in their closed positions. When starting with the jaws in the open state, pushing the cutting blade 410 through the space formed by the lower channel 406 and upper channel 408 towards the distal end of the treatment end 306 pushing surface 702 applies a force on the upper surface 706 in a downward direction, i.e., depicted by arrows D. Relatedly, pushing surface 704 applies a force on the lower surface 708 in an upward direction, i.e., depicted by arrow U. The arrows D and U on both sides of the blade portion B of the I-shaped cutting blade 410 is indicative that those forces can be applied on both sides of the central portion. In some embodiments, the lower jaw 404 is fixed and non-movable relative to the body of the treatment end 306 and/or the shaft 304, and the upper jaw 402 would be pushed downwards towards the lower jaw 404 using the rotating fulcrum 608 placed at the base end of the upper jaw 402 until the two jaws are closed together. In other embodiments, the upper jaw 402 is fixed and non-movable relative to the body of the treatment end 306 and/or the shaft 304, and the lower jaw 404 would be pushed upwards towards the upper jaw 402 using the rotating fulcrum 608 placed at the base end of the lower jaw 404 until the two jaws are closed together.

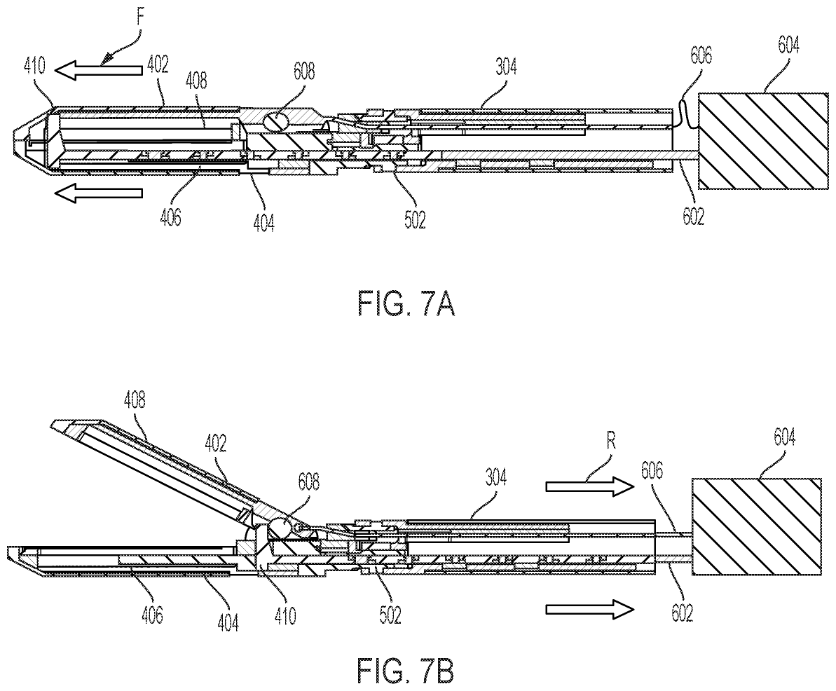

FIGS. 7A and 7B further illustrate the mechanism of opening and closing of upper jaw 402 relative to lower jaw 404. As shown in FIG. 7A, when the slider 604 pushes the cutting blade member 602, cutting blade 410 would be pushed forward from proximal end towards the distal end (as shown by arrow) of the treatment end 306 through the upper and lower channels, closing upper jaw 402 and the lower jaw 404. The downward pushing surface 702 initially contacts sloped surface 710 and gradually slides through the sloped surface 710 and the upper surface 704 by pushing downward the upper jaw 402 towards the lower jaw 404. The upper jaw pulling wire 606 is also simultaneously pushed into the shaft 304 using slider 604, forming loose tensions in the upper jaw pulling wire 606. As shown in FIG. 7B, when the slider 604 pulls back the cutting blade member 602, cutting blade 410 would be pulled back through the upper and lower channels from the distal end towards the proximal end of the treatment end 306 and is retracted (as shown by arrow R). The upper jaw pulling wire 606 will be simultaneously pulled back by the slider 604, opening the upper jaw 402 using the rotating fulcrum 608 placed at the base end of the upper jaw 402. Through the continued pulling back force through the pulling wire 606, sufficient opening strength of the upper jaw 402 would be realized, enabling the peeling functions required for the operator during the treatment process.

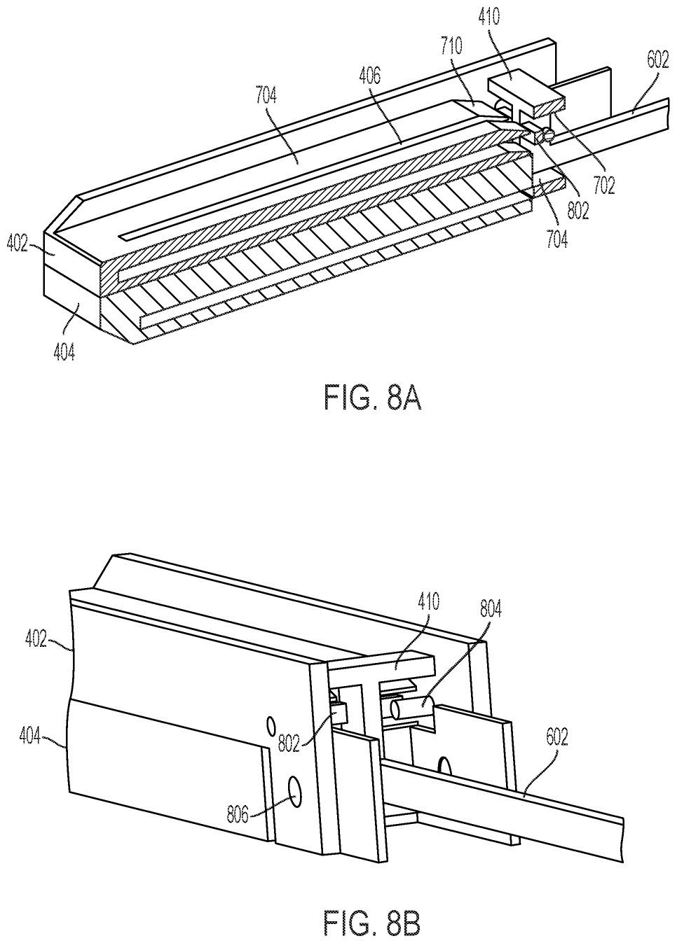

FIG. 8A and 8B illustrates another embodiment illustrating the mechanism of opening and closing of upper jaw 402 relative to lower jaw 404. As shown in FIG. 8A, when the cutting blade member 602 is pushed forward towards the distal end of the treatment end 306, the downward pushing surface 702 of the cutting blade 410 would push downward the upper surface 706, thereby closing the upper jaw 402 toward the lower jaw 404, in case the upper jaw 402 was in the open state. The downward pushing surface 702 initially contacts sloped surface 710 and gradually slides through the sloped surface 710 and the upper surface 704 by pushing the upper jaw 402 downwards towards the lower jaw 404. On the other hand, the hooking member 802 protruding from the cutting blade 410 would go through a channel formed beneath the upper surface 702, a channel being part of the upper channel 406. As shown in FIG. 8B, when the cutting blade 410 is pulled backward by the cutting blade member 602, the protruding member 802 being part of the upper jaw 402 comes in contact with the hooking member 804. The pulling back force of the cutting blade 410 together with the rotating motion achieved by the rotating fulcrum 806 placed at the lower jaw 404 would serve to rotate open the upper jaw 402 into the open state. Through the continued pulling back force through the cutting blade member 602, sufficient opening strength of the upper jaw 402 would be realized, enabling the peeling functions required for the operator during the treatment process. By coming in contact with the protruding member 802, the hooking member 804 would also stop the cutting blade 410 from pulling back too much into shaft 304. The resistance of the protruding member 802 felt through the movable handle 308 would inform the operator that the cutting blade 410 has reached the end point of its pulling back and would also inform the operator that the opening of the jaw 402 would start.

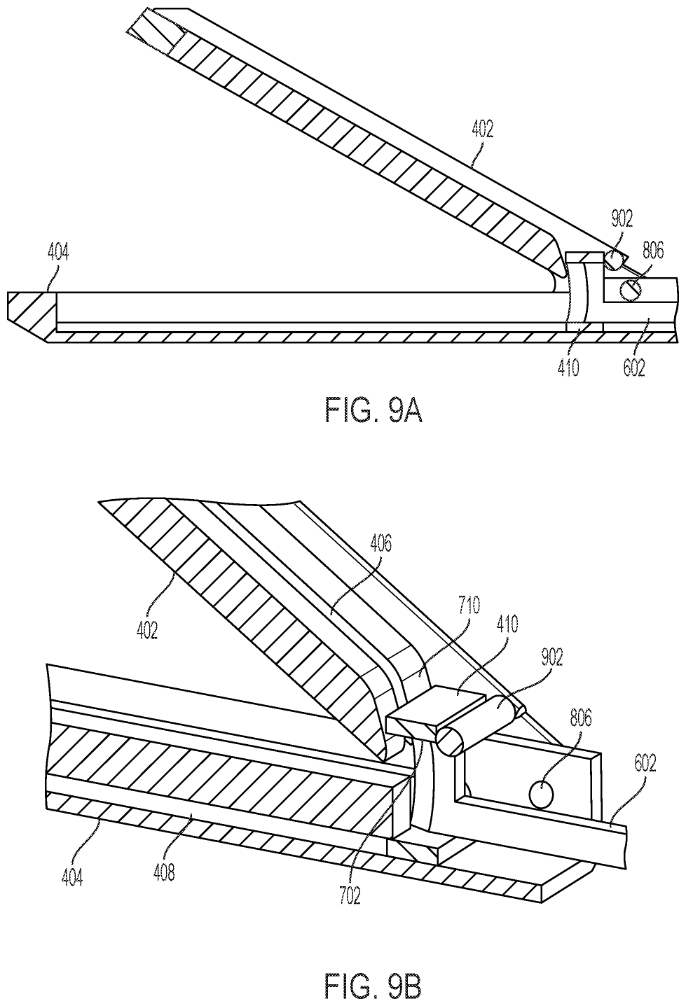

FIG. 9A and 9B illustrates another embodiment further illustrating the mechanism of opening and closing of upper jaw 402 relative to lower jaw 404. As shown in FIG. 9A, when the cutting blade member 602 is pushed backward towards the proximal end of the treatment end 306, the upper portion of the cutting blade 410 would make contact with the protruding member 902. Further pulling backward the cutting blade 410 together with the rotating motion achieved by the rotating fulcrum 806 placed at the lower jaw 404 would serve to rotate open the upper jaw 402 into the open state. Through the continued pulling back force through the cutting blade member 602, sufficient opening strength of the upper jaw 402 would be realized, enabling the peeling functions required for the operator during the treatment process.

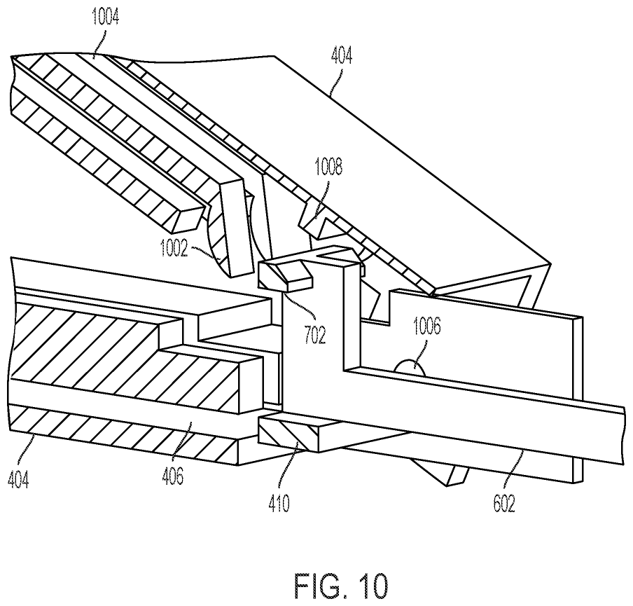

FIG. 10 illustrates another embodiment illustrating the mechanism of opening and closing of upper jaw 402 relative to lower jaw 404. The cutting blade 410 is pushed forward by the cutting blade member 602 through the upper and lower channels. The downward pushing surface 702 of the cutting blade 410 contacts the sloped surface 1002 and gradually pushes down the movable upper channel 1004. Because the movable upper channel 1004 is connected to the upper jaw 404, the downward force, together with the rotation movement of rotating fulcrum 1006, would push the upper jaw 402 downwards towards its closed state. The stopper 1008 serves to stop the movable upper channel 1004 from being pulled back towards the proximal direction of the treatment device 300 when the cutting blade 410 is pulled back for opening the upper jaw 402, as further described below.

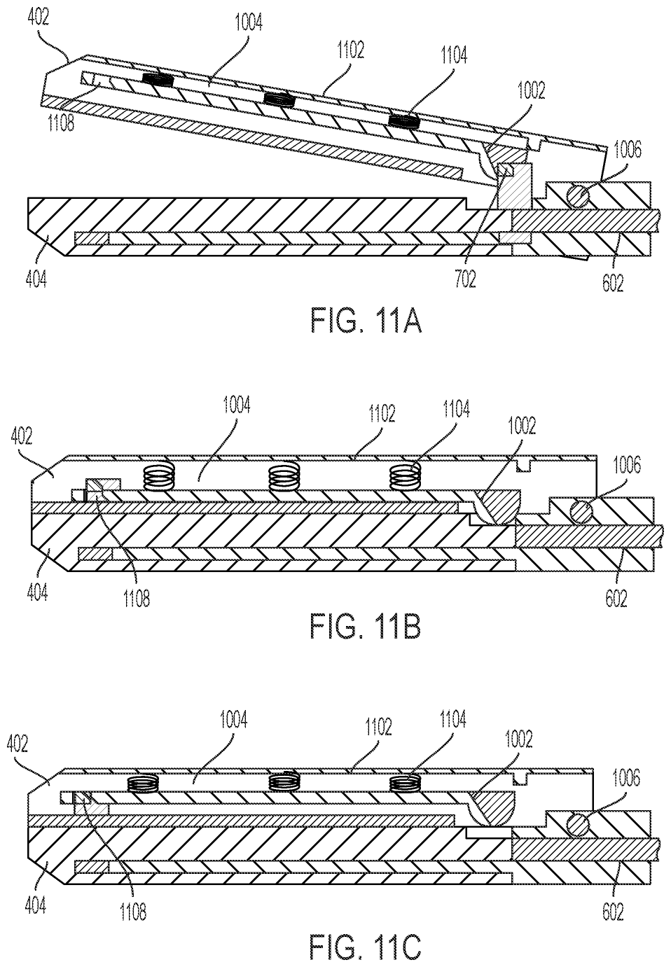

FIG. 11A, 11B, and 11C illustrates a cross sectional view of the upper and lower jaws and the mechanism of closing the upper jaw 402 relative to lower jaw 404 using a displaceable upper channel 1004. As shown in FIG. 11A, when the downward pushing surface 702 of the cutting blade 410 contacts and pushes downward the slope 1002, the displaceable upper channel 1004 receives a downward force. Because the displaceable upper channel 1004 is connected to the ceiling 1102 of upper jaw 402 through the elastic members 1104, the entire upper jaw 402 will be moved downward using the rotation movement of the rotating fulcrum 1006. The displaceable upper channel 1004 will be pulled downward within the upper jaw 402 using the expanding nature of the elastic members 1104, separated from the ceiling 1104. The elastic member 1104 can be a spring, a rubber band, or other elastic member allowing elongation and contraction. As shown in FIG. 11B, the displaceable upper channel 1004 would be pushed downward by the downward pushing surface 702 of the cutting blade 410, all the while the elastic member 1104 maintains the contracting force conveying upward bias to the movable upper channel 1004. As shown in FIG. 11C, when the downward pushing surface 702 of the cutting blade 410 reaches the distal end of the treatment end 306 and the opening 1108 of the displaceable upper channel 1004, the displaceable upper channel 1004 would be pulled upward by the contraction of the elastic member 1104 towards the ceiling 1102.

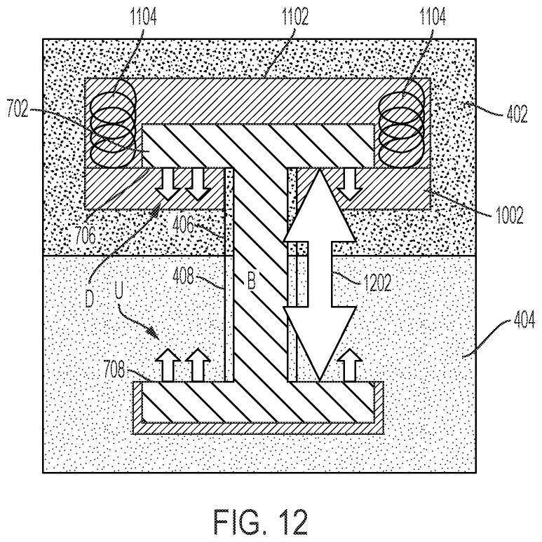

FIG. 12 illustrates the cross-sectional view of the upper and lower jaws in a closed state. The height 1202 of the blade portion B of the I-shaped cutting blade 410 is equivalent (within manufacturing tolerances) to the distance between the upper surface 706 of the upper channel 406 and the lower surface 708 of the lower channel 408 when the upper jaw 402 and lower jaw 404 are in their closed positions. The downward pushing surface 702 of the I-shaped cutting blade 410 pushes down the displaceable upper channel 1002, separating it away from the ceiling 1102 of the upper jaw 402. The elastic member 1104 would be expanded but maintains the contraction force that pulls the movable upper channel upward in the direction of the ceiling 1102.

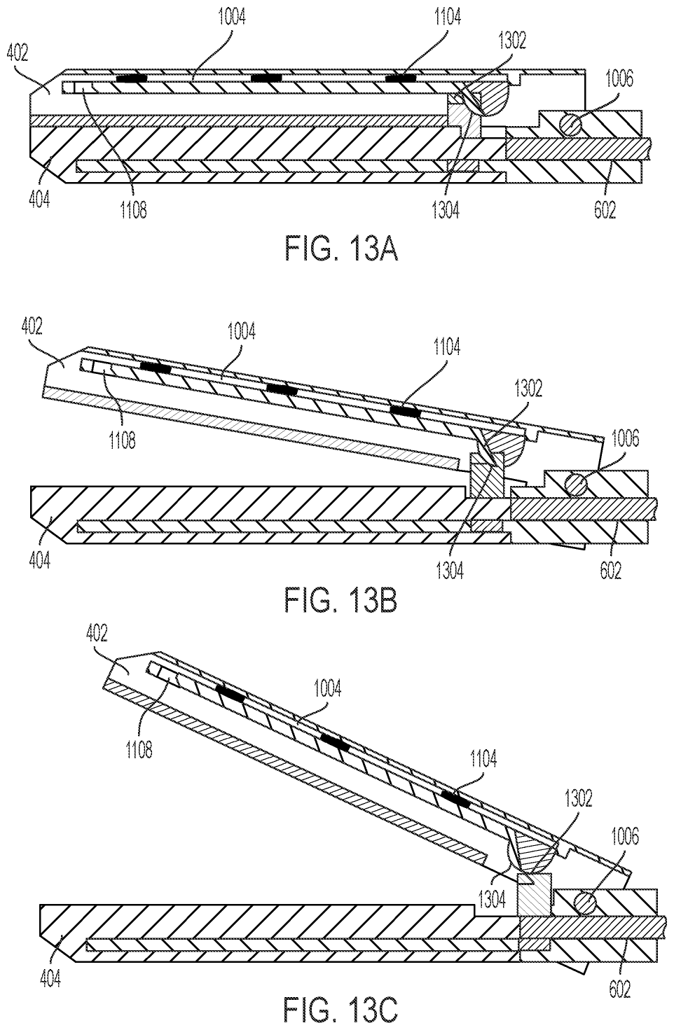

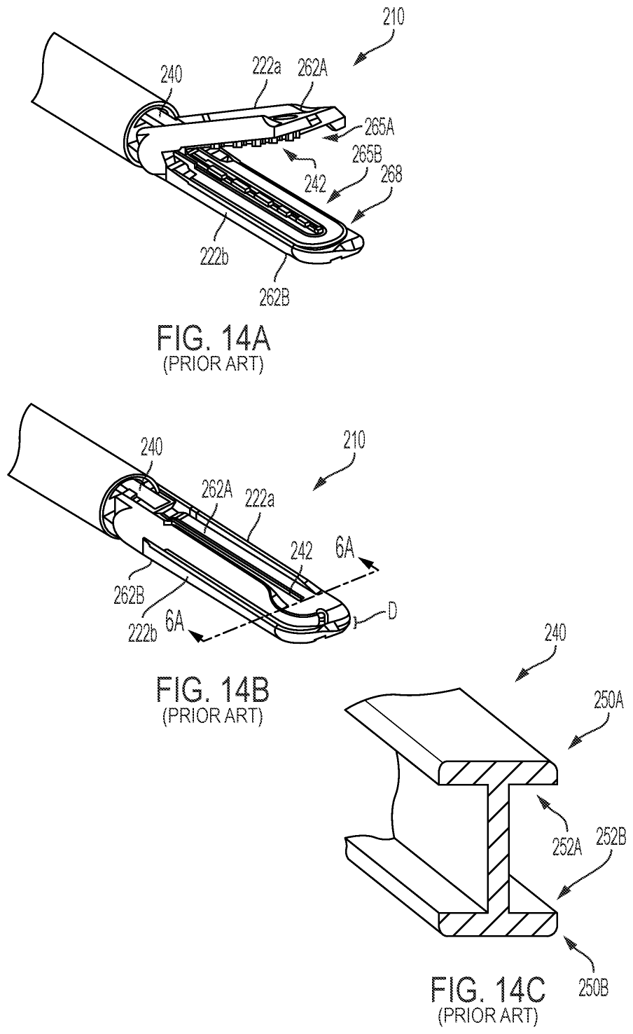

FIGS. 13A, 13B, and 13C illustrate the mechanism of opening the upper jaw 402 relative to lower jaw 404 using the displaceable upper channel 1004. As shown in FIG. 13A, when the cutting blade 410 is pulled backward away from the distal end of the treatment end 306, it will slide under the displaceable upper channel 1004 using the opening 1108 and the cutting blade sloped surface 1302 formed at the upper surface of the cutting blade 410. The cutting blade 410 would then slide beneath the displaceable upper channel 1002 until the cutting blade slope 1302 comes in contact with the curved surface 1304, which is part of the movable upper channel 1004. The curved surface can have any suitable curved shape, such as a semicircle, an oval or an ovid, both partial and full semicircle, oval and ovid shapes can be used. As shown in FIG. 13B, the cutting blade slope 1302 would contact and apply a force to the curved surface 1304, thereby pushing the displaceable channel 1004 upward. The entire upper jaw 402 would open upward as a result of such application of upward force and the rotation movement of the rotating fulcrum 1006. As shown in FIG. 13C, as the cutting blade 410 continues pulling backward in the proximal direction, the cutting blade slope 1302 would slide past the curved surface 1304 of the displaceable upper channel 1004 and the two parts would exchange its position. The position of the displaceable upper channel 1002 and the cutting blade 410 would revert back to pre-closing state described in FIG. 10A. Through the force resulting from the continued movement of the cutting blade member 602, sufficient opening force is applied to the upper jaw 402, enabling the peeling functions required for the operator during the treatment process.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A treatment device, comprising:
a body including a movable handle;
a longitudinally extending shaft having a proximal end connected to the body; and
a treatment end pivotally joined to a distal end of the shaft,
wherein the treatment end includes:
a movable cutting blade,
a jaw structure having a first jaw and a second jaw, the first jaw and the second jaw are connected at a base end by a joint structure to pivot the first jaw relative to the second jaw to open and close the jaw structure,
a first channel in the first jaw and a second channel in the second jaw, wherein with the jaw structure in a closed state, the first channel and the second channel form a route for a cutting blade to move back and forth longitudinally between the base end of the jaw structure and a distal end of the jaw structure, in response to a first input at the body, and
means to apply a force to move the first jaw away from the second jaw in response to a second input at the body.

2. The treatment device according to claim 1, wherein the means to apply the force to move the first jaw away from the second jaw in response to the second input at the body is a wire connected to one of the first jaw or the second jaw.

3. The treatment device according to claim 1, wherein the means to apply the force to move the first jaw away from the second jaw in response to the second input at the body is a hooking member protruding from the movable cutting blade in combination with a member protruding from one of the first jaw or the second jaw, and wherein, during a retraction of the movable cutting blade, the hooking member contacts the member protruding from one of the first jaw or the second jaw and applies the force to move the first jaw away from the second jaw.

4. The treatment device according to claim 1, wherein the means to apply the force to move the first jaw away from the second jaw in response to the second input at the body is a rear surface of the movable cutting blade in combination with a member protruding from one of the first jaw or the second jaw, and wherein, during a retraction of the movable cutting blade, the rear surface of the cutting blade contacts the member protruding from one of the first jaw or the second jaw and applies the force to move the first jaw away from the second jaw.

5. The treatment device according to claim 1, wherein the means to apply the force to move the first jaw away from the second jaw in response to the second input at the body is a member connected to the inner ceiling of one of the first jaw or the second jaw with an elastic member in combination with a sloped surface formed on the movable cutting blade and, wherein, during a retraction of the movable cutting blade, the sloped surface engages with the protruding from one of the first jaw or the second jaw to apply the force to move the first jaw away from the second jaw.

6. The treatment device according to claim 5, wherein the elastic member is a spring.

7. The treatment device according to claim 1, wherein the second input is a movement of the movable handle.

8. The treatment device according to claim 1, wherein the movable cutting blade includes a slope on its surface.

9. The treatment device according to claim 1, wherein a slider is placed between the movable handle and the first and second jaws.

10. The treatment device according to claim 1, wherein the body includes a connection to connect to a power source that is capable of supplying power for conducting a high-frequency treatment with the treatment device, and wherein at least one of the first jaw and second jaw serves as a high frequency electrode.

11. The treatment device according to claim 1, wherein the movable cutting blade is a different pole electrode compared to at least one of the first and second jaws.

12. The treatment device according to claim 1, wherein a joint structure is placed between the shaft and the first and second jaws.

13. The treatment device according to claim 12, wherein the joint structure includes at least a pulley or a link.

14. A treatment device, comprising:
a body including a movable handle;
a longitudinal extending shaft having a proximal end connected to the body; and
a treatment end pivotally joined to a distal end of the shaft,
wherein the treatment end includes:
a movable cutting blade, and
a jaw structure having a first jaw and a second jaw, the first jaw and the second jaw connected at a base end by a joint structure to pivot the first jaw relative to the second jaw to open and close the jaw structure, and
wherein the movable cutting blade includes:
a downward pushing surface and a upward pushing surface that close the jaw structure by sandwiching the first jaw and the second jaw while moving toward a distal side, and
a jaw pulling wire that applies a force to pull the first jaw away from the second jaw.

15. The treatment device according to claim 14, wherein the movable cutting blade is connected to a cutting blade member that transmits a driving force to close the jaw structure.

16. The treatment device according to claim 15, wherein the jaw pulling wire and the cutting blade member are connected to a slider, and the slider is linked to the movable handle.

17. A treatment device, comprising:
a body including a movable handle;
a longitudinal extending shaft having a proximal end connected to the body; and
a treatment end pivotally joined to a distal end of the shaft,
wherein the treatment end includes:
a movable cutting blade, and
a jaw structure having a first jaw and a second jaw, the first jaw and the second jaw connected at a base end by a joint structure to pivot the first jaw relative to the second jaw to open and close the jaw structure, and
wherein the movable cutting blade includes:
a downward pushing surface and a upward pushing surface that close the jaw structure by sandwiching the first jaw and the second jaw while moving toward a distal side, and
wherein the first jaw has a contact portion that comes into contact with the movable cutting blade when the movable cutting blade is moved in a proximal direction.

18. The treatment device according to claim 17, wherein the movable cutting blade includes a hooking member that comes into contact with the contact portion.

19. The treatment device according to claim 17, wherein the first jaw is provided with a slope surface that contacts the downward pushing surface.

20. The treatment device according to claim 17, wherein the movable cutting blade is connected to a cutting blade member that transmits a driving force to open and close the jaw structure.
